(19) United States
(12) Patent Application Publication
(10) Pub. No.: US 2004/0102823 A1
(43) Pub. Date: May 27, 2004

(54) Wax filled heating pad

(76) Inventor: Michael Schnoor, Eagan, MN (US)

Correspondence Address: US CORPORATIONS, INC., 630 NEPTUNE AVE., ENCINITAS, CA, 92024, US

(21) Appl. No.: 10/455,274

(22) Filed: Jun. 4, 2003

Related U.S. Application Data
(60) Provisional application No. 60/428,092, filed on Nov. 21, 2002.

(51) Int. Cl. A61F 7/00

(52) U.S. Cl. 607/96

(57) ABSTRACT

Warming devices are described having solid organic particulates that are solid at room temperature. The contents can be heated by a variety of methods such as microwave radiation or hot liquids. The solid particles act as a reusable heat reservoir to release heat over an extended period of time or absorb ambient heat and serve a protective function.

A generally flat bag to hold a quantity of paraffin wax and vegetable oil. The paraffin wax and oil can be heated in a microwave oven. Once heated, the wax holds a large quantity of heat and can produce heating for several hours. The wax can also be cooled in a freezer to produce hours of cooling therapy, if desired.

### CROSS-REFERENCE TO RELATED APPLICATION

This application relies on a previously filed provisional application 60,428,092 filed on Nov. 21, 2002.

### STATEMENT REGARDING FEDERALLY SPONSORED RESEARCH OR DEVELOPMENT

Not applicable

### REFERENCE TO SEQUENCE LISTING

Not applicable

### BACKGROUND OF THE INVENTION

1. Field of the Invention

This invention relates to heating pads and particularly to heating pads using a wax compound heating element.

2. Description of Related Art

Heating pads have been used for years to warm the body during cold weather and to help soothe sore muscles or joints after heavy work or exercise. Ice has also been used as a way of preventing inflammation and swelling of injured areas of a body. Recent developments in gel technology have produced a gel pack that can be heated in a microwave oven, or cooled in a freezer. This type of pack, however, must be wrapped in a towel before use, as direct contact with the skin can cause injury to the user. Moreover, the gel packs do not provide lasting heat. Finally, the gel packs can crack or break open. Once the plastic container has failed, the gel pack is useless as the gel cannot be contained.

This invention relates to heating pads which are used for heat therapy on the human body. Heating pads of various types have been used for many years. These heat devices are used to relieve pain and discomfort of sore muscles and joints. Water bottles, electric heat pads, corn filled packs and gel packs have all been used for heat therapy. All of these have their perspective merits and faults. In addition it may also be frozen and utilized as a cold pack.

The hot water bottle is ineffective; the electric heating pad requires electricity and limits mobility of the user, due to cord restraints. The corn filled packs produce an offensive odor and may mildew over time. The gel packs do not provide lasting heat and can crack open leaving it useless. We have found a viable solution to these problems. Our new invention uses a creative new substance, for the fill. It is a paraffin base heat pack. The paraffin mixture holds heat for an increased duration, and does not emit an odor. The pack is portable, microwaveable and is made for repetitive use.

The walls ( film) that encases the paraffin mix are microwave safe and will not melt. One of its benefits is that it can be micro waved repetitively. It may also be used repetitively as a cold pack whereby the paraffin pack is able to be placed directly in the freezer as necessary.

The physiological benefits of heat therapy are numerous. When heat is applies, the blood vessels expand which brings more circulation to the affected area. This allows for an increase of nutrients and oxygen at the cellular level and it aids in removing waste products that result from inflammation.

There is a temporary decrease in the amount of pain felt and this can increase the range of motion and benefits therapeutic exercises.

Paraffin heat therapy may aid in the treatment of muscle strain, arthritis, inflammation, muscle spasms and more. It is a drug-free and non-invasive method of applying heat to relieve joint stiffness and muscle pain.

### SUMMARY OF THE INVENTION

The paraffin filled heat pack is filled with food-grade paraffin. The paraffin mixture is completely safe and harmless substance. Paraffin heat packs are reusable and can retain heat for 2+ hours. You microwave it for a few minutes, and it may radiate heat for 2+ hours. This heat pack is portable with an outer bag and strap enclosure or cloth pad. This unique pack may be tailored for different sizes and shapes dependant upon the desired end use (Elbow, neck, leg, wrist, knee etc).

This invention is an improved, durable, reliable, multi-use heat pack. This paraffin heat pack is superior in providing heat due to its inventive wax blend material, giving it portability and extended heat retention. It will be especially useful for the home consumer, hospitals, nursing homes and physical therapy centers. It is convenient, affordable, comfortable and reliable alternative heat therapy. In addition, it may also be used as a cold pack if frozen.

The instant invention overcomes the problems listed above. It uses a generally flat bag to hold a quantity of paraffin wax. The paraffin wax can be heated in a microwave oven. Once heated, the wax holds a large quantity of heat and can produce heating for several hours. The corn can also be cooled in a freezer to produce hours of cooling therapy, if desired. The bag not only holds the wax, but also acts a thermal shield to protect the user from excessive temperatures produced by the wax.

Another advantage of the device is that they can be made in a variety of styles, thereby increasing the effectiveness of the therapy. For example, one style fits over a user's shoulders and neck. Another can be worn around the waist or the back. Another can be worn around the knee, ankle or elbow. Another model can be worn as a mitt to warm a user's hands.

It is an object of this invention to provide a heating and cooling pad that can be worn on a person to cover an affected area of the person's body to direct heat or cold into the affected area.

It is another object of this invention to provide a heating and cooling pad that automatically insulates the source of heat or cold from direct contact with a user's skin. There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new heating pad material which has many of the advantages heating pads mentioned heretofore and many novel features that result in a heating pad which is not anticipated, rendered obvious, suggested, or even implied by any of the prior art, either alone or in any combination thereof

It is another object of the present invention to provide a new heating pad material which may be easily and efficiently manufactured and marketed.

It is a further object of the present invention to provide a new heating pad material which is of a durable and reliable construction.

An even further object of the present invention is to provide a new heating pad material which is susceptible of a low cost of manufacture with regard to both materials and labor, and which accordingly is then susceptible of low prices of sale to the consuming public, thereby making such paraffin wax heating pad economically available to the buying public.

Still yet another object of the present invention is to provide a new paraffin wax heating pad which provides in the apparatuses and methods of the prior art some of the advantages thereof, while simultaneously overcoming some of the disadvantages normally associated therewith.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying descriptive matter in which there are illustrated preferred embodiments of the invention.

What is claimed as being new and desired to be protected is as follows:

1. A heating pad comprising:
a first and second wall having the same configuration and being in alignment therewith and means for fixedly connecting said first wall and said second wall together, thereby forming a bag structure which contains an inventive mixture of paraffin and vegetable oils.

2. A heating pad according to claim 1 wherein the device is used for heat therapy for pain relief

3. A heating pad according to claim 1 wherein the device is microwavable

4. A heating pad according to claim 1 wherein the device is reusable

5. A heating pad according to claim 1 wherein the device contains from 10% to 90%, paraffin in conjunction with misc vegetable oils.

6. A heating pad according to claim 1 wherein the device can be used in conjunction with a pouch or wrap

7. A heating pad according to claim 1 wherein the device may be used frozen or chilled to reduce swellings

8. A heating pad according to claim 1 wherein the device can be used frozen or chilled to keep food or drinks cold.

9. A heating pad according to claim 1 wherein the device can be constructed in different shapes and sizes to suit use on varying body parts. (Neck, arm, wrist, shoulder, elbow, knee, ankle, leg, back etc as desired by the consumer.

10. A heating pad according to claim 1 where in the device contains an optimal content of 80% paraffin and 20% vegetable oil (or any organic compound derivative and articles thereof)
